(19) United States
(12) Patent Application Publication
(10) Pub. No.: US 2015/0173661 A1
(43) Pub. Date: Jun. 25, 2015

(54) Medical Device Applicators

(71) Applicant: Abbott Diabetes Care Inc., Alameda, CA (US)

(72) Inventor: Christopher Ammon Myles, Alameda, CA (US)

(73) Assignee: Abbott Diabetes Care, Inc., Alameda, CA (US)

(21) Appl. No.: 14/416,614

(22) PCT Filed: Jul. 26, 2013
(86) PCT No.: PCT/US13/52397

§ 371 Date: Jan. 22, 2015

Related U.S. Application Data
(60) Provisional application No. 61/676,623, filed on Jul. 27, 2012.

(51) Int. Cl. A61B 5/153; A61B 5/145

(52) U.S. Cl. CPC A61B 5/153; A61B 5/14503; A61B 5/14532; A61B 2560/0406; A61B 2560/0462; A61B 5/1451

(57) ABSTRACT

On-body analyte monitoring devices configured for uncompressed and compressed configurations and methods of using the analyte monitoring devices are disclosed. The devices comprise a collapsible housing, wherein upon desired placement and user application of force to the housing converts the analyte monitoring device from an uncompressed configuration to a low-profile compressed state while guiding an analyte sensor through the skin and into contact with bodily fluid to measure and analyte level therein. Also provided are systems and kits.

### PRIORITY

This application claims priority to U.S. Provisional Patent Application Ser. No. 61/676,623, filed on Jul. 27, 2012, entitled “Medical Device Applicators”, the disclosure of which is incorporated herein by reference in its entirety for all purposes.

### BACKGROUND

Diabetes Mellitus is an incurable chronic disease in which the body does not produce or properly utilize insulin. Insulin is a hormone produced by the pancreas that regulates blood sugar (glucose). In particular, when blood sugar levels rise, e.g., after a meal, insulin lowers the blood sugar levels by facilitating blood glucose to move from the blood into the body cells. Thus, when the pancreas does not produce sufficient insulin (a condition known as Type 1 Diabetes) or does not properly utilize insulin (a condition known as Type 2 Diabetes), the blood glucose remains in the blood resulting in hyperglycemia or abnormally high blood sugar levels.

One element of managing blood glucose levels is testing to monitor blood glucose levels. Conventional in vitro techniques, such as drawing blood samples, applying the blood to a test strip, and determining the blood glucose level using colorimetric, electrochemical, or photometric test meters may be employed. Another technique for monitoring glucose levels is by using an in vivo glucose monitoring system that continuously or automatically tests glucose. Such in vivo glucose monitoring systems utilize a wholly implanted or partially implanted sensor that measures glucose in bodily fluid, such as blood or interstitial fluid during the time that it is implanted in the body. One example of such a system is the FreeStyle Navigator® Continuous Glucose Monitoring System manufactured by Abbott Diabetes Care Inc.

In in vivo glucose monitoring systems, a user inserts at least a portion of an in vivo glucose sensor under or through the skin. Typically, an applicator assembly is employed to insert the sensor in the body of the user. For insertion, a needle engaged with the sensor pierces the skin of the user and is then removed from the body of the user while leaving the sensor in place. Separate from the applicator assembly, sensor electronics are attached to the surface of the user's body above the sensor.

It is desirable that the insertion of an analyte sensor is relatively easy to perform and only involves minimal pain for the patient. Ease of use is particularly important because users of analyte sensors may have physical limitations due to age and/or disease. Accordingly, analyte sensor applicators are described, including integrated devices that integrate an analyte sensor, a disposable applicator, and a sensor electronics into an all-in-one unit.

### SUMMARY

Some embodiments of the present disclosure include a housing comprising a top, a base and a plurality of levels, the housing having an extended configuration and a compressed configuration, wherein the levels are lockable with one another in the compressed configuration, and an applicator that includes a needle for insertion through the user's skin when the base of the housing is on the skin and the housing is depressed to transition the device from the extended configuration to the compressed configuration. In some embodiments, the base includes an adhesive patch or mates with a mount that has an adhesive patch. Some embodiments include an analyte sensor positioned within at least a portion of the needle. The needle may include a lumen or may include a slot that extends along at least a portion of the needle. In some embodiments, the applicator including the needle is removable from around the sensor and out of the housing. In some embodiments, the housing levels include a plurality of rings. In some embodiments, the rings are adapted to interlock with pawl and notch features. In some embodiments, the plurality of rings includes three rings, wherein a first ring locks to a second ring and the second ring locks to a third ring. In some embodiments, the housing comprises a bi-stable bellows. In some embodiments, the top is provided by a top cap to the bellows. In some embodiments, the cap contains sensor electronics.

Some embodiments of the present disclosure include a method of inserting an analyte sensor including providing an applicator including a needle mated with an analyte sensor housed therein, placing a housing containing the applicator against the skin of a user, adhering the housing to the skin, depressing the housing to transition it from an extended configuration to a compressed configuration, wherein depressing the housing causes the applicator to protrude from the housing through the skin, inserting the mated analyte sensor through the skin, and removing the applicator including the needle, leaving the housing adhered to the skin and the analyte sensor through the skin. Some embodiments include locking the housing in the compressed configuration upon depression. In some embodiments, depressing the housing to the compressed configuration includes telescoping rings of the housing into one another. In some embodiments, depressing the housing to the compressed configuration includes flexing a bellows of the housing into the compressed configuration. In some embodiments, the bellows lock in a compressed configuration by virtue of a bi-stable design configuration.

Some embodiments of the present disclosure include an analyte monitoring device including a collapsible housing having an extended position and a compressed position, the housing including sensor electronics; and an integrated, removable applicator including a needle having a separable analyte sensor operable to communicate with the sensor electronics, wherein the needle of the applicator is recessed within the housing when the housing is in the extended position and protrudes from a lower surface of the housing when the housing is in the compressed position. In some embodiments, the applicator is removable from the housing when the housing is in the compressed position and wherein the analyte sensor separates from the needle when the applicator is removed. In some embodiments, the analyte monitoring device further includes a releasable lock that prevents the housing from transitioning between the extended position and the compressed position. In some embodiments, the analyte monitoring device activates when the housing transitions from the extended position to the compressed position. In some embodiments, the analyte monitoring device activates when the applicator is removed from the housing.

Numerous other aspects and embodiments are provided. Other features and aspects of the present invention will become more fully apparent from the following detailed description, the appended claims, and the accompanying drawings.

### INCORPORATION BY REFERENCE

Patents, applications and/or publications described herein, including the following patents, applications and/or publications are incorporated herein by reference for all purposes: U.S. Pat. Nos. 4,545,382, 4,711,245, 5,262,035, 5,262,305, 5,264,104, 5,320,715, 5,356,786, 5,509,410, 5,543,326, 5,593,852, 5,601,435, 5,628,890, 5,820,551, 5,822,715, 5,899,855, 5,918,603, 6,071,391, 6,103,033, 6,120,676, 6,121,009, 6,134,461, 6,143,164, 6,144,837, 6,161,095, 6,175,752, 6,270,455, 6,284,478, 6,299,757, 6,338,790, 6,377,894, 6,461,496, 6,503,381, 6,514,460, 6,514,718, 6,540,891, 6,560,471, 6,579,690, 6,591,125, 6,592,745, 6,600,997, 6,605,200, 6,605,201, 6,616,819, 6,618,934, 6,650,471, 6,654,625, 6,676,816, 6,730,200, 6,736,957, 6,746,582, 6,749,740, 6,764,581, 6,773,671, 6,881,551, 6,893,545, 6,932,892, 6,932,894, 6,942,518, 7,041,468, 7,167,818, 7,299,082, 7,618,369, 7,630,748 and 7,866,026, U.S. Patent Publication Nos. 2004/0186365, 2005/0182306, 2006/0025662, 2006/0091006, 2007/0056858, 2007/0068807, 2007/0095661, 2007/0108048, 2007/0199818, 2007/0227911, 2007/0233013, 2008/0066305, 2008/0081977, 2008/0102441, 2008/0148873, 2008/0161666, 2008/0172205, 2008/0267823, 2009/0054748, 2009/0294277, 2010/0213057, 2010/0081909, 2009/0247857, 2011/0106126, 2011/0082484, 2010/0326842, 2010/0198034, 2010/0324392, 2010/0280782, 2010/0234710, 2010/0230285, 2010/0313105, 2011/0213225, 2011/0021889, 2011/0193704, 2011/0190603, and 2011/0191044, U.S. patent application Ser. Nos. 13/071,461, 13/071,487, and 13/071,497, and U.S. Provisional Application Nos. 61/325,260, 61/563,744 and 61/569,287.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiments or examples of the present subject matter in whole or in part.

FIG. 1A is a perspective view of an analyte monitoring device with a housing in an extended configuration according to some embodiments of the present invention.

FIG. 1B is a perspective view of an analyte monitoring device with a housing in a compressed configuration according to some embodiments of the present invention.

FIG. 1C is a perspective view of an analyte monitoring device with a housing in a compressed configuration with an applicator removed according to some embodiments of the present invention.

FIG. 2A is a perspective view of an analyte monitoring device with an alternate housing in an extended configuration according to some embodiments of the present invention.

FIG. 2B is a perspective view of an analyte monitoring device with an alternate housing in a compressed configuration according to some embodiments of the present invention.

FIG. 2C is a perspective view of an analyte monitoring device with an alternate housing in a compressed configuration with an applicator removed according to some embodiments of the present invention.

FIG. 3 is a flow chart illustrating an example method of inserting an analyte sensor according to some embodiments of the present invention.

### DETAILED DESCRIPTION

On-body analyte sensor devices are disclosed. Some embodiments of these devices comprise a collapsible housing, wherein upon desired placement and user application of force, the housing transitions from an extended configuration to a low-profile compressed configuration while guiding a sensor into subcutaneous contact with an analyte to be monitored. A removable applicator including a needle may be provided to facilitate such insertion. Alternatively, the devices described above may exchange the sensor for an infusion needle or catheter and the subject device may function as an infusion set.

Regarding such hardware, in some embodiments, an on-body analyte sensor device is provided that incorporates applicator features and function. The device is applied to (or by) a user in an extended configuration. When an amount of force above a predetermined threshold is applied to the top of the device, features shift/give-way allowing the device to compress. Such action may be accomplished by overrunning detent features or by the structure to be compressed/altered/converted as further described below.

In a sensor-insertion embodiment, as the device configuration changes as it collapses/compresses, an included applicator needle assists in inserting the sensor. After insertion into the skin, the on-body analyte sensor device is maintained in its low-profile collapsed/compressed configuration. This configuration can be locked in place by mechanical features and/or the back of an adhesive skin patch. The applicator needle may be then removed from the device (e.g., popped out via spring features and/or is removed manually by the user) and is disposed of safely.

In reference to sensor-based embodiments, these may include various configurations as described in any of U.S. Patent Application Ser. Nos. 61/569,287 filed Dec. 11, 2011, Ser. No. 12/698,129 filed Feb. 1, 2010, Ser. No. 13/071,461 filed Mar. 24, 2011, Ser. No. 13/071,487 filed Mar. 24, 2011, Ser. No. 13/071,497 filed Mar. 24, 2011 and Ser. No. 13/407,750 filed Feb. 28, 2012, each of which are incorporated by reference in their entirety. Embodiments of sensor configurations are described in any of U.S. patent application Ser. No. 12/131,012 filed May 30, 2008, Ser. No. 12/714,439 filed Feb. 26, 2010 and Ser. No. 12/842,013 filed Jul. 22, 2010, U.S. Pat. No. 7,497,827 to Brister et al. issued Mar. 3, 2009, each of which are incorporated by reference in their entirety. Any included sensor applicator needle may likewise be as described in any of the referenced applications. Otherwise it may be configured as shown and described in further detail below. Regardless, it is specifically contemplated that alternative constructions suitably used in connection with the present disclosures may be drawn from any of the patent filings cited in the above INCORPORATION BY REFERENCE section.

Still, in some embodiments, the applicator needle used to insert the sensor may be associated with a spring-loaded shuttle or other means to drive refraction. In other embodiments, the applicator needle is manually withdrawn after sensor insertion is complete. In such embodiments, mechanical or structural features are advantageously provided to lock the applicator needle to the housing of the sensor device until the installation action is complete. Such mechanical or structural features may include one or more deflectable arms with locking bosses or tangs (e.g., pawls) that are displaced from complimentary locking (e.g., catch/recess) features. Otherwise, the features may act as gripping detent features that locate position but are overcome by adequate retraction force (for example, by user finger grip and withdrawal).

Regardless of whether configured for sensor placement or infusion set installation, various housing configurations are possible. The following two examples are provided but other embodiments are possible.

In one example, a plurality of telescoping barrels or ring sections is provided. A significantly reduced height is achieved when the rings telescope into one another. Unlike a collapsible drinking cup however, the present invention includes sensor electronics and the applicator. Still further, in some embodiments, the sensor device includes a mounting base with an adhesive layer affixed thereto as well as a closed cap as further described below. Emplaced/installed on a user, the device is thus closed-off. Still further, latch or locking features are advantageously provided to lock the various collapsible rings or levels together once actuated. These features may be in the form of deflectable arms with bosses or tangs (e.g., pawls) that lock with complimentary catch/recess/interdental features or other related means.

In further embodiments, a cap incorporating the sensor elements (e.g., sensor associated electronics, battery, etc.) is operatively connected to a type of bellows. Unlike the bellows arrangement found in a typical bellows pump dispenser, the bellows incorporated in this embodiment of the present disclosure is configured to remain either expanded in a fully extended configuration or collapsed in a fully compressed configuration. Thus, it may be regarded as having a “bi-stable” design. As such, in the extended configuration, the bellows may provide resistance to be overcome in compressing the device, requiring a threshold force to collapse the housing and drive-in the applicator needle with the sensor. Moreover, such a device remains locked in position with the sensor in place when driven to its fully collapsed state and a facing surface of the device base is adhered to a user's skin by an included adhesive patch or section.

In either embodiment (e.g., telescoping rings or bi-stable bellows) and either form (e.g., analyte sensor or infusion set), the present invention simplifies the insertion of an analyte sensor/infusion set by including an integrated applicator in an all-in-one device. Instead of having to first attach a device to the user, then attach an applicator to the device, then trigger the applicator, and then replace the applicator with sensor/infusion electronics, the present device includes both an applicator and sensor/infusion electronics within the housing of the device which are triggered when the device is placed on the user and compressed. The applicator can then be removed and safely disposed.

Such methods include placing an adhesive base of a (sensor or infusion set) device against a user's skin, and pressing on the device's housing to compress it, the force overcoming a threshold level of resistance and driving a needle into the skin while compressing the device. Where, upon compression, the device achieves a low profile and is adapted to remain locked in the low-profile configuration. The lock may be irreversible (i.e., without breaking lock features), or may simply be adequate to maintain position if not pulled-on. In one method, the device is actuated with pieces sliding or telescoping into one another. In another method, the device is actuated with pieces stacking onto one another in accordion fashion.

Variations of the apparatus and methods may further include removing the application (including the needle) from the device, if one is provided for use in sensor introduction. Likewise, variation of the apparatus and methods may include locking elements or portions of elements of the device together to maintain a compressed configuration. Still further, variations of the apparatus and methods may include precedent acts of removing the subject device from sterile packaging, removing any protective cover or peel ply from an included adhesive element and/or assembling or activating any incorporated electronics—manually or automatically/consequently in association with the other actions.

In any case, further details and features of the subject hardware and methods of use may be appreciated in reference to the patent filings cited in the above INCORPORATION BY REFERENCE section from which features may be drawn. Still further, upon review of the subject disclosure those with skill in the art may appreciate other variations within the spirit or scope of the subject inventions. The appended claims are intended to cover such variations directly or by way of equivalents. Unless expressly excluded by virtue of the subject claim language, all such equivalents are meant to be captured irrespective of any specific recital herein. Aspects of the present disclosure include the subject devices, kits in which they are included, and their methods of use and manufacture.

Before the present disclosure is described in detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

Embodiments of the present disclosure include analyte monitoring devices configured to be worn on the body of a user, the devices including an analyte sensor configured for placement through a skin layer for measuring the concentration level of an analyte, such as glucose, of the user. In some embodiments, the analyte sensor is configured for subcutaneous placement through the skin for contacting a biological fluid of the user, such as interstitial fluid or the user's blood stream and may be wholly positioned under the skin or may be transcutaneously positioned so that a portion of the sensor is below the skin and a portion is above. In some embodiments, the analyte sensor is inserted through the user's skin layer by way of an applicator, which may include a needle with a sharp tip to facilitate insertion through the user's skin. Signals generated by the analyte sensor in contact with the user's biological fluid are transmitted to the on-body analyte monitoring device and converted to data corresponding to the user's analyte levels, such as a blood glucose level. In some embodiments, the data is transmitted to a receiver or reader unit and the user's analyte level is displayed to the user. Various systems for measuring and displaying a user's analyte level are disclosed in, for example, among others, U.S. Pat. Nos. 6,175,175, 6,565,509, and 6,560,471, and in U.S. Publication Nos. 2010/0198034 and 2011/0213225, the disclosures of each of which are incorporated herein by reference for all purposes.

In some embodiments, an analyte monitoring device may be configured to facilitate insertion of an analyte sensor without the use of an applicator. In such embodiments, the sensor may include a sharp tip so that the sensor can be pushed through the skin without a needle.

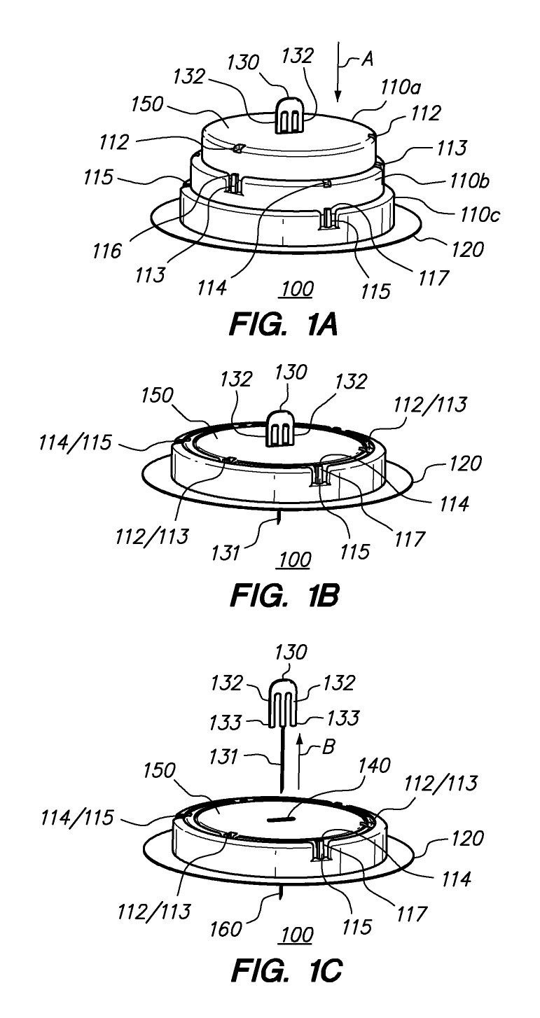

Referring now to the figures, FIGS. 1A to 1C depict an analyte monitoring device including an analyte sensor and an integrated but removable applicator. FIG. 1A depicts the analyte monitoring device 100 in an uncompressed or extended configuration, i.e., in a pre-insertion state. FIG. 1B depicts the analyte monitoring device in a collapsed configuration, i.e., inserted state, and FIG. 1C depicts the analyte monitoring device 100 in a wearable configuration with the applicator 130 removed.

In some embodiments, analyte monitoring device 100 includes sections defining a plurality of levels 110a, 110b, 110c. The levels may be rings. Levels 110a, 110b, 100c telescope or nest into one another or nest together when a downward force is applied on the analyte monitoring device 100. As can be seen in FIGS. 1A and 1B, upon depression of the analyte monitoring device 100 in a downward direction (represented by arrow A) by the application of the downward force (for example, manual force by a user) on the device top cap or cover 150, level 110a telescopes within level 110b, which in turn telescopes within level 110c. In some embodiments, analyte monitoring device 100 may include a locking mechanism to prevent depression of the analyte monitoring device 100 until the lock is released, thus preventing accidental depression. While only three levels are shown in the configuration of FIGS. 1A to 1C, it is understood that the analyte monitoring device 100 may include any number of levels including two, four or five or more levels. Together, the rings that define the levels 110a, 110b, 110c, and cap 150 define the overall device housing.

Referring back to FIGS. 1A and 1B, the levels 110a, 110b, 110c include a plurality of interlocking features configured and arranged to lock together upon depression of the analyte monitoring device 100. As shown, level 110a includes recesses 112 (or referred to as notch or catch), which are arranged with corresponding deflectable arms 113 with tang or boss 116 (together optionally referred to as a pawl) on the next level 110b. Upon depression of the analyte monitoring device 100, deflectable arms 113 with tang or boss 116 lock into recesses 112, thus locking level 110a within level 110b. Similarly, level 110b includes recesses 114, which are arranged with corresponding deflectable arms 115 with tang or boss 117 on level 110c, which lock together upon depression of the analyte monitoring device 100. In this manner, the analyte monitoring device 100 is stabilized for on-body use on the skin surface during the use time period.

It is understood that a variety of shapes or configurations of the recess/arm locking mechanism may be employed, such as, recesses, notches or catches that correspond to arms with tangs or bosses together referred to as pawls. It is further understood that other locking mechanisms may be employed including, but not limited to, latches, snaps, buttons, or links or friction or magnetic locking mechanisms. In some embodiments, a single locking mechanism may lock together all levels of the analyte monitoring device.

Referring back to FIGS. 1A to 1C, also shown is applicator 130. Applicator 130, in some embodiments, is configured to mate with an analyte sensor 160, to facilitate insertion of the analyte sensor through the skin of the user. By converting from the expanded housing configuration in FIG. 1A (e.g., via user applied downward force) to the collapsed housing configuration in FIG. 1B, the applicator 130 is driven to protrude past the base (whereas it was previously contained fully within the multilevel housing) so that it can penetrate the skin at an insertion site. FIG. 1C illustrates removal of applicator 130. It may be manually removed by overcoming outward bias of arms 132 with boss or tang features 133 with edges of a slot 140 in cap 150.

Applicator 130, in some embodiments, includes a needle 131 with a sharp distal end having a width suitable to pierce the skin of the subject while causing minimal pain or discomfort. Applicator 130 may also include a user engageable proximal end that is constructed for grasping, e.g., a handle. The handle may be enlarged relative to the distal end. In the embodiment of FIG. 1C, the handle includes arms 132. Arms 132 provide sufficient width to allow the user to grasp the applicator 130 to assist with applicator removal (as shown in FIG. 1C). The needle 131 of the applicator 130 and analyte sensor 160 are mated such that depression of the analyte monitoring device 100 causes the applicator 130 and mated analyte sensor 160 to protrude from the base of the device 100 and to be inserted through the skin of the user. Thereafter, removal of the applicator 130 from the user, while maintaining the analyte sensor 160 in the inserted position is enabled. In some embodiments, the applicator 130 may be of uniform size and width over the length of the applicator 130. In other embodiments, analyte sensor 160 may include a sharp tip and may be configured for insertion through the skin of the user without the aid of an applicator 130.

As can be seen in the progression of FIGS. 1A to 1C, depression of analyte monitoring device 100 in the downward direction A, in some embodiments, causes the levels 110a,110b,110c, of analyte monitoring device 100 to telescope within each other and, in some embodiments, lock into place in a compressed state. When in the uncompressed state, shown in FIG. 1A, the needle 131 of applicator 130 is housed entirely within the analyte monitoring device 100. However, upon depression and locking of analyte monitoring device 100, the needle 131 of applicator 130 extends beyond the base of the analyte monitoring device 100. In use, when the analyte monitoring device 100 is placed on the body of the user, and secured thereto, by, for example, an adhesive mounting unit 120, depression of analyte monitoring device 100 causes the needle 131 of the applicator 130 and mated analyte sensor 160 to be inserted through the skin of the user. In some embodiments, adhesive mounting unit 120 may include a hole or area therein to allow passage therethrough of the needle 131 of the applicator 130 and analyte sensor 160. In some embodiments, the length of the needle 131 of the applicator 130 and height of the housing of the analyte monitoring device 100 are selected such that the depth at which the applicator 130 is inserted upon depression of the analyte monitoring device 100 is fixed to a depth allowing the mated analyte sensor 160 to contact a patient's interstitial fluid. In other embodiments, the length of the applicator 130 may be adjustable. In still other embodiments, the height of the compressed analyte monitoring device 100 may be adjustable for use with a plurality of different length applicators and/or sensors.

Once the analyte monitoring device 100 is depressed and locked in position, the applicator 130 may be removed in the opposing direction (arrow B) as shown in FIG. 1C, leaving behind the analyte sensor 160 through the skin of the user and in contact with the user's interstitial fluid. In some embodiments, slit 140 is provided in the analyte monitoring device 100, which is sized and shaped to allow for passage therethrough by the applicator 130. In some embodiments, the slit 140 is configured with stops to keep the applicator 130 from entering too far into the analyte monitoring device 100. In other embodiments, the slit 140 includes a locking mechanism to lock the applicator 130 in place and require manipulation of the analyte monitoring device 100 and/or the applicator 130 to unlock and ultimately remove the applicator 130 from the analyte monitoring device 100. In some embodiments, the analyte monitoring device 100 and applicator 130 may be configured such that only a small hole, i.e., a needle pin hole is required. Except for such slit features, the cap 150 is essentially closed.

In some embodiments, analyte monitoring device 100 may include functionality of an infusion device. In such embodiments, the applicator may facilitate infusion of a therapeutic substance, such as medication including, for example insulin, to the user.

Referring back to FIGS. 1A to 1C, analyte monitoring device 100 may include electrical components (not shown) housed therein. For example, in the top or central level 110a of analyte monitoring device 100 sensor electronics may be disposed. Such components may include a power source, such as a battery, a processor and memory or other circuitry, such as an application specific integrated circuit (ASIC), a radio frequency (RF) transmitter and/or receiver and/or transceiver, electrical contacts, and an antenna. Some embodiments may include radio frequency identification (RFID) components and circuitry. Details of the electrical components of an analyte monitoring device are described in, among others, U.S. Pat. Nos. 6,175,175, 6,565,509, and 6,560,471, and in U.S. Publication Nos. 2010/0198034 and 2011/0213225, the disclosures of each of which are incorporated herein by reference for all purposes. In some embodiments, an antenna may be built into the interior and/or exterior of one or more of the levels of the analyte monitoring device. In some embodiments, top or central level 110a, housing the electrical components may be a solid or self-contained piece and lower levels 110b,110c, may be ring shaped, such that the center of levels 110b,110c are hollow to allow nesting or seating therein of top level 110a when the analyte monitoring device 100 is in the compressed state.

In some embodiments, analyte monitoring device 100 may further include a switch or other activation mechanism to activate analyte monitoring device 100 upon depression of the analyte monitoring device 100 into the compressed state. For example, contacts may be provided within the analyte monitoring device 100, such that upon depression of analyte monitoring device 100 and telescoping of the levels 110a, 110b, 110c together, the contacts touch and activate analyte monitoring device 100. In some embodiments, locking of the arms and recesses of the levels of analyte monitoring device 100 may engage a switch to activate analyte monitoring device 100. In some embodiments, removal of the applicator 130 may activate the analyte monitoring device 100. In other embodiments, a switch may be provided on the exterior of analyte monitoring device 100. In still other embodiments, activation of analyte monitoring device 100 may be achieved by a command sent from a receiver or reader device.

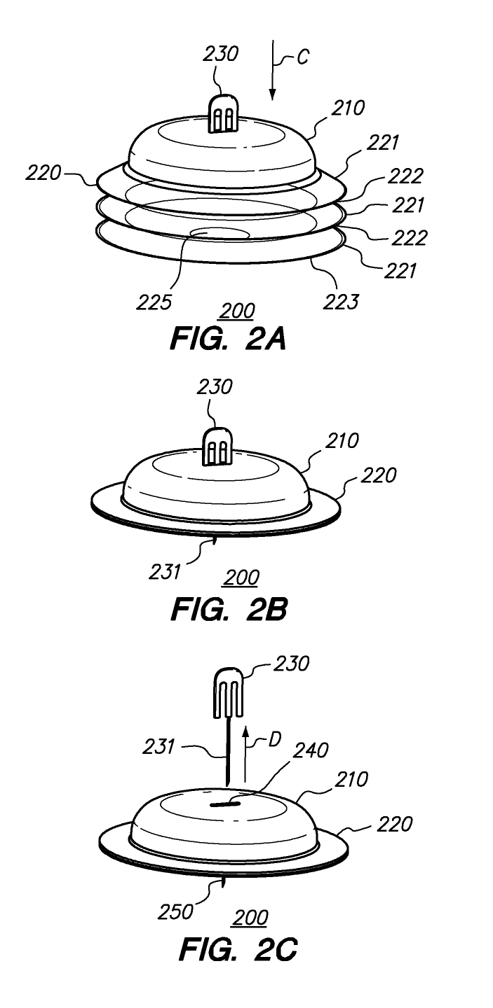

FIGS. 2A to 2C illustrate another embodiment of an analyte monitoring device according to the present invention. Analyte monitoring device 200 may include a top or cap section 210 housing electrical components (not shown) therein. Such components may include a power source, such as a battery, a processor and memory or other circuitry, such as an application specific integrated circuit (ASIC), a radio frequency (RF) transmitter and/or receiver and/or transceiver, contacts, and an antenna. Analyte monitoring device 200 may further include a bellows section 220 in some embodiments. Cap section 210 may be connected to the top end of bellows section 220, which is in an uncompressed configuration prior to sensor insertion, as shown in FIG. 2A.

Referring still to FIG. 2A, bellows section 220 comprises a plurality of levels or folds, each including raised portions 221 and fold portions 222. In some embodiments, raised portions 221 include seams where each level of the bellows is connected. In some embodiments, the bottom of the lowest level of the bellows section 220 may include an adhesive 223 for adhering the analyte monitoring device 200 to the skin of the user. In some embodiments, the adhesive 223 may be a concentric circle around the edge of the bottom layer of the bellows section 220. In other embodiments, the entire bottom surface of the analyte monitoring device 200 may be an adhesive 223. In some embodiments, an aperture 225 may be provided through the adhesive 223 to allow for the analyte sensor 250 and applicator 230 to pass therethrough.

Referring still to FIGS. 2A to 2C, similarly to the embodiments described in conjunction with FIGS. 1A to 1C, analyte monitoring device 200 also includes an applicator 230 with a needle 231 having a sharp distal end to facilitate insertion of an analyte sensor 250. In the uncompressed state shown in FIG. 2A, in some embodiments, the needle 231 of applicator 230 is housed entirely within the analyte monitoring device 200, and upon compression of the bellows section 220 of the analyte monitoring device 200, the applicator 230 and associated analyte sensor 250 are extended down beyond the base of the analyte monitoring device 200 and through the skin of the patient (see FIG. 2B). In some embodiments, only the bellows section 220 compresses upon force applied in the downward direction (represented by arrow C), while the cap 210 is rigid and does not compress or change configuration. In some embodiments, a locking mechanism may be provided to prevent accidental compression of analyte monitoring device 200 until the locking mechanism is disengaged.

Upon compression, bellows section 220 has a minimal height dimension, as illustrated in FIG. 2B. In some embodiments, upon compression, bellows section 220 is configured to retain its compressed state or configuration. That is, bellows section 220 is configured to be stable in two configurations (compressed or uncompressed), i.e., bi-stable. In some embodiments, a locking mechanism is provided to keep the bellows section 220 in the compressed state. In some embodiments, an adhesive may additionally or alternatively be used to keep the analyte monitoring device 200 in the compressed state. In some embodiments, the adhesive may be in the form of a double-sided adhesive 223 whereby upon compression, the bottom of cap 210 may be held to adhesive 223 via the double-sided adhesion. In other embodiments, each fold 222 of bellows section 220 may include an adhesive, such that upon compression and subsequent touching of the top and bottom of folds 222, the folds 222 stick together, thus keeping the bellows section in a compressed state.

Referring back to FIG. 2C, upon compression of analyte monitoring device 200, applicator 230 may be removed via slit 240 in the opposite direction (arrow D) from compression and insertion, leaving analyte sensor 250 implanted within the skin of the user. In some embodiments, compression of analyte monitoring device 200 may activate analyte monitoring device 200, for example, via an activation switch which is activated upon contact of cap section 210 with adhesive 223.

In some embodiments, after compression of analyte monitoring device 100/200 (FIGS. 1A to 1C and 2A to 2C), analyte monitoring device 100/200 may have a height or thickness profile of approximately 10 mm or less, e.g., about 4 mm or less, above the skin layer.

While the embodiments shown in FIGS. 1A to 1C and 2A to 2C illustrate a circular shaped analyte monitoring device, it is understood that the device may include other shapes, such as, a square, rectangle, triangle, oval, or any polygon or non-polygon shape. Furthermore, while the embodiments shown illustrate insertion of only a single analyte sensor, it is also contemplated that the device may include two or more analyte or other sensors configured for insertion upon compression of the analyte monitoring device. In such embodiments, the applicator 130/230 may include more than one needle and/or the device may include more than one applicator 130/230. In some embodiments, the analyte monitoring device may include additional components including, but not limited to, a display, input buttons, skin temperature sensors or ambient temperature sensors.

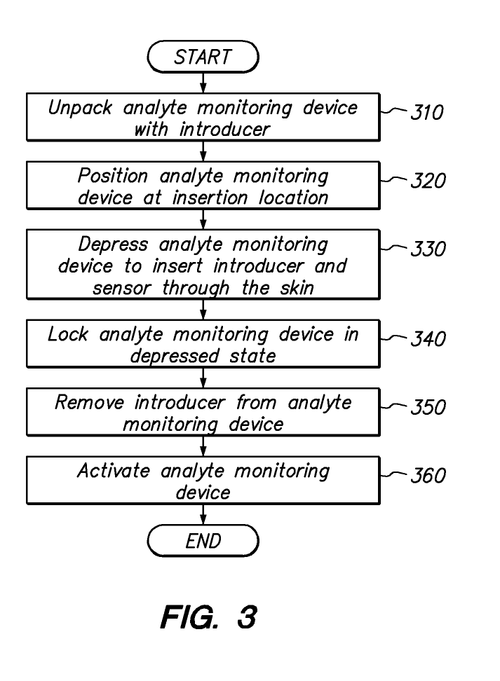

FIG. 3 is a flow chart illustrating an example of analyte sensor insertion according to some embodiments of the present disclosure. Referring to FIG. 3, a user unpacks the analyte monitoring device (310), such as analyte monitoring device 100 as illustrated in FIGS. 1A to 1C, which includes a sensor applicator 130. While the description herein is with reference to the analyte monitoring device 100 shown in FIGS. 1A to 1C, is it understood that such method may be applicable to a variety of other analyte monitoring device configurations such as those described in conjunction with FIGS. 2A to 2C and also in U.S. Patent Application Ser. Nos. 61/569,287 filed Dec. 11, 2011, Ser. No. 12/698,129 filed Feb. 1, 2010, Ser. No. 13/071,461 filed Mar. 24, 2011, Ser. No. 13/071,487 filed Mar. 24, 2011, Ser. No. 13/071,497 filed Mar. 24, 2011 and Ser. No. 13/407,750 filed Feb. 28, 2012, each of which are incorporated by reference in their entirety.

In some embodiments, analyte monitoring device 100 is originally housed and shipped in sterile packaging. In some embodiments, analyte monitoring device 100 is packaged as a single unit including electronics, applicator, housing, and sensor. In other embodiments, electronic components and/or an analyte sensor may require separate unpacking. In embodiments wherein either the electronic components and/or analyte sensor are packaged separately, the analyte monitoring device is assembled with the electronic components and/or analyte sensor to place the analyte monitoring device in condition for ready use.

Upon completion of unpacking, the analyte monitoring device 100 is placed at an insertion location (320) on the skin of the user. In some embodiments, the insertion location may include one or more of the user's abdomen, thigh, or arm. The analyte monitoring device 100 is depressed so as to insert the applicator 130 and mated analyte sensor 160 through the skin of the user (330) at the insertion site. In some embodiments, analyte monitoring device 100 may need to be unlocked prior to depression and subsequent insertion of the applicator 130 and analyte sensor 160. In some embodiments, depression of the analyte monitoring device 100 may further result in adhesion of an adhesive layer 120 to the skin of the user to keep analyte monitoring device 100 in place. The amount of force required for depression of analyte monitoring device 100 may be tuned to specific requirements based on the material and/or configuration of the analyte monitoring device 100 and any locking mechanisms. In some embodiments, the analyte monitoring device 100 is configured to alert, via an audio, visual or tactile alert, when the analyte monitoring device 100 has been correctly and completely depressed.

Once depressed, the analyte monitoring device 100 is locked in the depressed state (340) with analyte sensor 160 inserted through the skin of the user and in contact with the user's bodily fluid, such as intestinal fluid. In some embodiments, locking of the analyte monitoring device 100 in the depressed state is automatic. In other embodiments, the user may need to manually lock the analyte monitoring device 100 in the depressed state. Once locked in the depressed state with analyte sensor 160 inserted through the skin of the user, applicator 130 is removed from analyte monitoring device 100, while leaving the analyte sensor 160 in contact with the user's bodily fluid (350).

Referring back to FIG. 3, analyte monitoring device 100 may then be activated (360) for monitoring the user's analyte, such as glucose levels. In some embodiments, analyte monitoring device 100 is configured to be active for 3 days or more, such as 5 days or more, 7 days or more, 14 days or more or one month or more. In some embodiments, active time of analyte monitoring device 100 may be based on the active lifetime of sensor 160. In other embodiments, active time of analyte monitoring device 100 may be based on a battery life of analyte monitoring device 100. In some embodiments, analyte monitoring device 100 may activate automatically upon depression of analyte monitoring device 100 and/or upon detection of removal of applicator 130. In some embodiments, activation is done by the user by, for example, activating a switch, removing a power-off locking mechanism, or by bringing a receiver or reader device to within a predetermined distance of analyte monitoring device 100. Further details regarding activation and use of analyte monitoring systems can be found in, for example, among others, U.S. Publication Nos. 2010/0198034 and 2011/0213225, the disclosures of each of which are incorporated herein by reference for all purposes.

Within the scope of the present disclosure, the number of levels described in the embodiments above may be varied. At least one telescoping or collapsible level will be provided. As many as four or more may be provided as well when deeper insertion of a longer needle may be desired.

Some embodiments of the present disclosure include a housing comprising a top, a base and a plurality of levels, the levels having an extended configuration and a compressed configuration, wherein the levels are lockable with one another in the compressed configuration, and an applicator including a needle for insertion through the skin of a user upon placement of the base of the housing on the skin and depressing the housing to convert the device from the extended configuration to the compressed configuration.

In some embodiments, the base includes an adhesive patch. Some embodiments include an analyte sensor positioned within at least a portion of the needle of the applicator. In some embodiments, the applicator (including the needle) is removable from around the sensor and out of the housing. In some embodiments, the housing levels comprise a plurality of rings. In some embodiments, the rings are adapted to interlock with pawl and notch features. In some embodiments, the plurality of rings includes three rings, wherein a first ring locks to a second ring and the second ring locks to a third ring. In some embodiments, the housing comprises a bi-stable bellows. In some embodiments, the top is provided by a top cap to the bellows. In some embodiments, the cap contains sensor electronics.

Some embodiments of the present disclosure include a method of inserting an analyte sensor comprising providing a device having an applicator including a needle mated with an analyte sensor housed therein, placing the device against the skin of a user, adhering the device to the skin, depressing the device to convert it from an extended configuration to a compressed configuration, wherein depressing the device inserts the needle of the applicator and mated analyte sensor through the skin, and removing the applicator including the needle, leaving the device adhered to the skin and the analyte sensor through the skin.

Some embodiments include locking the device in the compressed configuration upon depression. In some embodiments, depressing the device to the compressed configuration includes telescoping rings of the device into one another. In some embodiments, depressing the device to the compressed configuration includes flexing a bellows of the device into the compressed configuration. In some embodiments, the bellows locks in a compressed configuration by virtue of a bi-stable design configuration.

Some embodiments of the present disclosure include a method of monitoring an analyte comprising providing an analyte monitoring device, the analyte monitoring device including a top portion and a base portion, wherein the analyte monitoring device includes an uncompressed state configuration and a compressed state configuration, providing an applicator housed within the analyte monitoring device when in the uncompressed state, providing an analyte sensor mated with the applicator, adhering the analyte monitoring device to a skin surface of a user, depressing the analyte monitoring device, wherein depressing the analyte monitoring device inserts the applicator and mated analyte sensor through the skin layer of the user, removing the applicator including the needle from the analyte monitoring device, wherein upon removing the applicator, the analyte sensor remains inserted through the skin, and activating the analyte monitoring device.

In some embodiments, the top portion of the analyte monitoring device includes electronic components. In some embodiments, the base portion of the analyte monitoring device includes a plurality of ring portions, and wherein depressing the analyte monitoring device includes telescoping the plurality of ring portions within each other. In some embodiments, the base portion of the analyte monitoring device includes a bellows section, and wherein depressing the analyte monitoring device includes compression of the bellows section. Some embodiments include locking the analyte monitoring device in the compressed state configuration. Some embodiments include releasing a lock of the analyte monitoring device prior to depressing the analyte monitoring device. In some embodiments, activating the analyte monitoring device is automatic upon depressing the analyte monitoring device. In some embodiments, activating the analyte monitoring device is automatic upon removal of the applicator. Some embodiments include monitoring an analyte level of the user for an active lifetime of the analyte sensor. In some embodiments, the active lifetime of the analyte sensor is 14 days or more.

As used above and in the appended claims, the singular forms “a”, “an”, and “the” include plural referents unless the context clearly dictates otherwise. Reference to a singular item includes the possibility that there is a plurality of the same items present. In other words, use of the articles allow for “at least one” of the subject item in the description above as well as the claims below. When two or more items (for example, elements or processes) are referenced by an alternative “or”, this indicates that either could be present separately or any combination of them could be present together except where the presence of one necessarily excludes the other or others.

As will be apparent to those of skill in the art upon reading this disclosure, each of the individual embodiments or variations described and illustrated herein has discrete components and features which may be readily separated from or combined with the features of any of the other several embodiments without departing from the scope or spirit of the present disclosed subject matter. Any recited method can be carried out in the order of events recited, or in any other order which is logically possible. Likewise, it is contemplated that any particular feature of the inventive variations described may be set forth and claimed independently, or in combination with any one or more of the features described herein. As such, the claims may be drafted to exclude any optional element. Accordingly, this statement is intended to serve as antecedent basis for use of such exclusive terminology as “solely,” “only” and the like in connection with the recitation of claim elements, or use of another type of “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

Where a range of values is provided, it is understood that each intervening value between the upper and lower limit of that range and any other stated or intervening value in that stated range, is encompassed within the disclosed subject matter. Every range stated is also intended to specifically disclose each and every “subrange” of the stated range. That is, each and every range smaller than the outside range specified by the outside upper and outside lower limits given for a range, whose upper and lower limits are within the range from said outside lower limit to said outside upper limit (unless the context clearly dictates otherwise), is also to be understood as encompassed within the disclosed subject matter, subject to any specifically excluded range or limit within the stated range. Where a range is stated by specifying one or both of an upper and lower limit, ranges excluding either or both of those stated limits, or including one or both of them, are also encompassed within the disclosed subject matter, regardless of whether or not words such as “from”, “to”, “through”, or “including” are or are not used in describing the range.

Although any methods and materials similar or equivalent to those described herein can also be used in the practice or testing of the present disclosed subject matter, this disclosure may specifically mention certain exemplary methods and materials. All publications mentioned in this disclosure are, unless otherwise specified, incorporated herein by reference for all purposes, including without limitation to disclose and describe the methods and/or materials in connection with which the publications are cited.

The publications discussed herein are provided solely for their disclosure prior to the filing date of the present application. Nothing herein is to be construed as an admission that the present disclosed subject matter is not entitled to antedate such publication by virtue of prior invention. Further, the dates of publication provided may be different from the actual publication dates, which may need to be independently confirmed.

Nothing contained in the Abstract or the Summary should be understood as limiting the scope of the disclosure, however, they may be cited for the purpose of claim support. The Background, Abstract and the Summary are provided for bibliographic and convenience purposes and due to their formats and purposes should not be considered comprehensive.

The breadth of the present disclosure is not to be limited to the examples provided and/or the subject specification, but rather only by the scope of the claim language. Although the foregoing invention has been described in detail for purposes of clarity of understanding, it is contemplated that certain modifications may be practiced within the scope of the appended claims.

Certain embodiments may include a housing including a top, a base and a plurality of levels, the housing having an extended configuration and a compressed configuration, wherein the levels are lockable with one another in the compressed configuration, and a removable applicator disposed within the housing and including a needle wherein the needle moves from being recessed within the housing when the housing is in the extended configuration to protruding from the base when the housing is in the compressed configuration.

In certain aspects, the base may include an adhesive.

Certain embodiments may further include an analyte sensor disposed within at least a portion of the needle.

In certain aspects, the needle may be separable from the sensor when the needle protrudes from the base.

In certain aspects, each level of the plurality of levels may have different widths.

In certain aspects, each level of the plurality of levels may include at least one ring and each ring has a diameter different than every other ring.

In certain aspects, the rings may interlock together with pawl and notch features.

In certain aspects, the rings may include three rings, wherein a first ring locks to a second ring and the second ring locks to a third ring.

In certain aspects, the housing may include sensor electronics.

In certain aspects, the housing may include a bi-stable bellows.

In certain aspects, the top may include a cap to the bellows.

In certain aspects, the cap may include sensor electronics.

Certain embodiments may include a method of inserting an analyte sensor comprising placing an analyte monitoring device against the skin of a user, the analyte monitoring device including a removable applicator, the applicator including a needle, the needle being mated to an analyte sensor, and depressing the analyte monitoring device to convert it from an extended configuration to a compressed configuration to insert the needle and mated analyte sensor through the skin and cause the analyte monitoring device to lock in the compressed configuration.

Certain embodiments may further include removing the applicator from the analyte monitoring device and leaving the analyte monitoring device adhered to the skin and the analyte sensor through the skin.

In certain aspects, the depressing may be accomplished with a single motion.

In certain aspects, depressing the applicator to the compressed configuration may include telescoping rings of the analyte monitoring device into one another.

In certain aspects, depressing the analyte monitoring device to the compressed configuration may include flexing a bellows of the analyte monitoring device into the compressed configuration.

In certain aspects, the bellows may lock in a compressed configuration by virtue of a bi-stable design configuration.

Certain embodiments may include a collapsible housing having an extended position and a compressed position, the housing including sensor electronics, and an integrated, removable applicator including a needle having a separable analyte sensor operable to connect with the sensor electronics, wherein the needle of the applicator is recessed within the housing when the housing is in the extended position and protrudes from a lower surface of the housing when the housing is in the compressed position.

In certain aspects, the applicator may be removable from the housing when the housing is in the compressed position and wherein the analyte sensor separates from the needle when the applicator is removed.

Certain embodiments may further include a lock that prevents the housing from transitioning between the extended position and the compressed position.

In certain aspects, the analyte monitoring device may activate when the housing transitions from the extended position to the compressed position.

In certain aspects, the analyte monitoring device may activate when the applicator is removed from the housing.

Various other modifications and alterations in the structure and method of operation of this disclosure will be apparent to those skilled in the art without departing from the scope and spirit of the embodiments of the present disclosure. Although the present disclosure has been described in connection with particular embodiments, it should be understood that the present disclosure as claimed should not be unduly limited to such particular embodiments. It is intended that the following claims define the scope of the present disclosure and that structures and methods within the scope of these claims and their equivalents be covered thereby.

1. A device, comprising:
a housing including a top, a base and a plurality of levels, the housing having an extended configuration and a compressed configuration, wherein the plurality of levels are lockable with one another in the compressed configuration; and
a removable applicator disposed within the housing and including a needle, wherein the needle moves from being recessed within the housing when the housing is in the extended configuration to protruding from the base when the housing is in the compressed configuration.

2. The device of claim 1, wherein the base includes an adhesive.

3. The device of claim 1, further comprising an analyte sensor disposed within at least a portion of the needle.

4. The device of claim 3, wherein the needle is separable from the analyte sensor when the needle protrudes from the base.

5. The device of claim 1, wherein each level of the plurality of levels has different widths.

6. The device of claim 1, wherein each level of the plurality of levels includes at least one ring, and each ring has a diameter different than every other ring.

7. The device of claim 6, wherein the at least one ring includes a plurality of rings that interlock together with pawl and notch features.

8. The device of claim 6, wherein the plurality of rings include three rings, wherein a first ring locks to a second ring and the second ring locks to a third ring.

9. The device of claim 1, wherein the housing includes sensor electronics.

10. The device of claim 1, wherein the housing includes a bi-stable bellows.

11. The device of claim 10, wherein the top includes a cap to the bi-stable bellows.

12. The device of claim 11, wherein the cap includes sensor electronics.

13. A method of inserting an analyte sensor, comprising:
placing an analyte monitoring device against a skin surface, the analyte monitoring device including a removable applicator, having a needle mated to the analyte sensor;
depressing the analyte monitoring device to convert it from an extended configuration to a compressed configuration to insert the needle and the mated analyte sensor through the skin surface and to lock the analyte monitoring device in the compressed configuration.

14. The method of claim 13, further comprising removing the removable applicator from the analyte monitoring device and leaving the analyte monitoring device adhered to the skin surface and the analyte sensor through the skin surface.

15. The method of claim 13, wherein depressing the analyte monitoring device to the compressed configuration is accomplished with a single motion.

16. The method of claim 13, wherein depressing the removable applicator to the compressed configuration includes telescoping rings of the analyte monitoring device into one another.

17. The method of claim 13, wherein depressing the analyte monitoring device to the compressed configuration includes flexing a bellows of the analyte monitoring device into the compressed configuration.

18. The method of claim 17, wherein the bellows locks in the compressed configuration by virtue of a bi-stable design configuration.

19. An analyte monitoring device, comprising:
a collapsible housing having an extended position and a compressed position, the housing including sensor electronics; and
an integrated, removable applicator including a needle having a separable analyte sensor operable to connect with the sensor electronics, wherein the needle is recessed within the housing when the housing is in the extended position, and protrudes from a lower surface of the housing when the housing is in the compressed position.

20. The analyte monitoring device of claim 19, wherein the applicator is removable from the housing when the housing is in the compressed position and wherein the analyte sensor separates from the needle when the applicator is removed.

21. The analyte monitoring device of claim 19, further comprising a lock that prevents the housing from transitioning between the extended position and the compressed position.

22. The analyte monitoring device of claim 19, wherein the analyte monitoring device activates when the housing transitions from the extended position to the compressed position.

23. The analyte monitoring device of claim 19, wherein the analyte monitoring device activates when the applicator is removed from the housing.
